(19) United States
(12) Patent Application Publication
(10) Pub. No.: US 2026/0305797 A1
(43) Pub. Date: Oct. 8, 2026

(54) SKINCARE SYSTEM AND A METHOD OF USE

(71) Applicant: Andrew Georgiou, Wilmington, DE (US)

(72) Inventor: Andrew Georgiou, Wilmington, DE (US)

(21) Appl. No.: 19/529,928

(22) Filed: Feb. 4, 2026

Related U.S. Application Data
(63) Continuation-in-part of application No. 19/171,830, filed on Apr. 7, 2025.

(51) Int. Cl. A45D 44/00; A61Q 19/00; A61Q 19/08

(52) U.S. Cl. CPC A45D 44/00; A61Q 19/007; A61Q 19/08; A45D 2044/007

(57) ABSTRACT

A two-part skincare system for formulating a customized skin care formulation each time of application. The system includes a base moisturizer dispenser and a series of booster serum dispensers. The base moisturizer dispenser includes a base moisturizer, which acts as an emollient and carrier. Each of the series of functional booster serum dispensers includes booster serum. The different booster serums include different active ingredients for skincare function, such as anti-aging and firming. The user can select the serum and quantity of serum for their specific skin type and requirement.

### CROSS-REFERENCE TO RELATED APPLICATIONS

This application is a continuation of U.S. patent application Ser. No. 19/171,830, filed on Apr. 7, 2025. The entire disclosures of each of the foregoing applications are hereby incorporated by reference in their entirety.

### FIELD OF INVENTION

The present invention relates to skincare products, and more specifically, the present invention relates to skin care formulation that can be customized according to an individual's skincare requirement.

### BACKGROUND

Skin care products include a wide range of formulations used to clean, protect, and improve skin. Moisturizers are one of the most widely used formulations in skin care products. The basic purpose of moisturizers is to soothe the skin, providing protection and enhancement. Moisturizers are modified using different compositions for different skincare needs. However, the composition of moisturizers or modifications made to basic moisturizers depends on the skin type of the user. Currently, skin types are broadly classified into a set of categories, such as sensitive skin, dry skin, normal skin, and oily skin. The skin care products are formulated according to these standard skin types and made available in the market.

In the last decade, there has been a strong demand from customers for specialized skin care products. The customers have become more cautious about their skin health and desire skincare products that specifically suit their skin types and requirements. Moreover, individuals experience daily fluctuations in skin conditions due to environmental factors, hormonal changes, diet, or seasonal shifts. However, currently, commercially available skincare formulations are fixed and pre-measured. These products are packaged and sold as single-use formulations with static ingredient concentrations, offering no opportunity for the user to adjust, customize, or modulate the intensity or focus of their skincare regimen. The user can only vary the volume of a product applied, yet this does not alter the ratio or potency of the active ingredients. It merely increases or decreases the overall quantity of the product applied, without delivering true customization of active ingredient intensity or benefit.

Thus, there is an unmet and long-term need for a skincare system that allows a user to adapt their regimen in real time to address their specific skin types and periodic variations in the skin condition due to a variety of factors.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a two-part skincare system that can be customized in near real time for personalized skincare users' needs.

Another object of the present invention is that the two-part skincare system provides for skin protection and improvement.

Still another object of the present invention is that the two-part skincare system is easy to use.

Yet another object of the present invention is that an accurate amount of formulation can be dispensed.

A further object of the present invention is that the two-part skincare system is versatile in use.

In one aspect, disclosed is a two-part skincare system is disclosed that allows a fully customizable, adaptive skincare regimen that offers flexibility, personalization, and improved efficacy over traditional fixed formulations. By empowering the user to select and control the formulation each time it is applied, this system represents a significant advancement in personalized skincare.

In one aspect, disclosed is a two-part skincare system that includes a base moisturizer formulation and a series of functional booster serums. The base moisturizer may be designed as a universal carrier for hydration, nourishment, and skin barrier support. The series of functional Booster Serums, each formulated with active ingredients that deliver specific skincare functions, such as hydration, brightening, calming, or firming. In use, a user dispenses a portion of the base moisturizer and adds one or more drops of a selected functional Booster serum directly into this portion. The two components are blended manually by the user immediately prior to application. This allows the user to adjust both the type and concentration of active ingredients applied to the skin at each use.

In one aspect, disclosed is a two-part skincare system for formulating a customized skin care formulation each time of application. The system includes a base moisturizer dispenser and a series of functional booster serum dispensers. The base moisturizer dispenser includes a base moisturizer, which acts as an emollient and carrier. Each of the series of functional booster serum dispensers includes booster serums. The different booster serums include different active ingredients for skincare function, such as anti-aging and firming. The user can select the serum and quantity of serum for their specific skin type and requirement.

In one aspect, the user can determine the intensity of the final product by determining the volume of each element every time they apply it.

### BRIEF DESCRIPTION OF DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a flowchart showing the use of the disclosed two-part skincare system, according to an exemplary embodiment of the present invention.

FIG. 2 shows a booster serum dispenser, according to an exemplary embodiment of the present invention.

FIG. 3 shows components of the booster serum dispenser, according to an exemplary embodiment of the present invention.

FIG. 4 illustrates the steps of using the disclosed two-part skin care system, according to an exemplary embodiment of the present invention.

FIG. 5 illustrates an exploded view of the booster serum dispenser, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an”, and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention, since the scope of the invention will be best defined by the allowed claims of any resulting patent.

The invention described pertains to a two-part skin care system that can be customized as per the skin care needs of an individual. The disclosed skin care system can dispense customized formulations for each application, thus making the invention versatile and highly adaptable. The user can customize their skin care regimen almost daily depending upon changing factors, such as climate, hormonal balances, and the like. Thus, disclosed is a specialized skincare product that can be personalized and customized at individual levels, and for each application at an individual level.

The disclosed skin care system may include a base moisturizer and a booster serum. The base moisturizer can be universal and may be suitable for most skin types, and may provide basic hydration, skin conditioning, and barrier protection. The moisturizer may include main ingredients such as Collagen, Bakuchiol, and Peptides to provide general skin health support. The combination of Collagen, Bakuchiol, and Peptides may also stimulate deep collagen production, and enhance firmness & resilience. These characteristics of the base serum may be universal for all skin types, and thus, the base moisturizer can be used universally as a base or carrier with beneficial effects.

The base moisturizer may include various ingredients such as oils, petrolatum, humectants, and various excipients known to a skilled person. Also, any suitable process can be used for the formulation of the moisturizer, and any such method is within the scope of the present invention. The moisturizer may contain universal emollients only and not any active ingredients for the skin.

The booster serums may include active ingredients for different skin benefits. These may typically be concentrated formulations that can provide specific skin improvement. For example, the active ingredients may be treating or improving various skin conditions, such as aging, sun damage, skin irritation, skin breakout, skin blemishes, oily skin, and various other skin conditions known in the art. The active ingredients may also provide for protection and healing, such as nighttime skin rejuvenation and repair, daytime protection, and damage protection. An antiaging booster serum may slow down visible signs of aging and may include vitamins and antioxidants. Similarly, for dry and irritated skin, the serum can soothe and comfort the skin. The amount of serum can be increased or decreased based on the level of dryness and irritation. A person can use different serums at different times. For example, a nighttime repair serum can be used while sleeping for skin repair. A serum for daytime can protect the skin from damage due to the sun and harsh weather. Serum may also be used for specific skin conditions, such as oily skin, acne, and skin blemishes.

The moisturizer may be packed in a wide-mouth container. The consistency of the moisturizer may be thick, cream-like, and can be easily taken out in the desired quantity from a wide-mouth container. The wide-mouth container may include a flat cap at the top covering the opening. The flat cap may have a central hole. The container may also include a pump mechanism for drawing the base moisturizer. The pump mechanism may be in fluid communication with the central hole. The top cap may be pressed to actuate the pump mechanism, which deposits a predefined quantity of the base moisturizer on the top cap. The deposited base moisturizer can be taken by rubbing a finger or a spatula through the top cap.

The Booster Serum can be packed in specialized containers that may allow dispensing an accurate amount each time. Each functional booster serum may be contained in a separate container, enabling the user to select one or more serums at each application to achieve the desired skincare effect. The serum may preferably be of liquid consistency and can be dispensed as drops. The container of the serum may dispense the drops slowly so that the user may have control over the number of drops dispensed. The packaging of the serum may include a label providing information like the type of serum, its use, and recommended quantity.

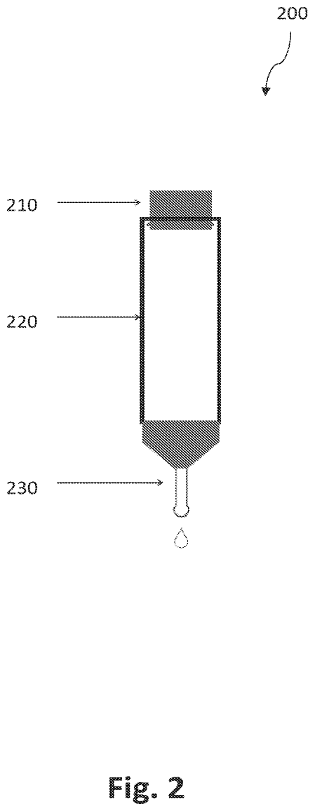

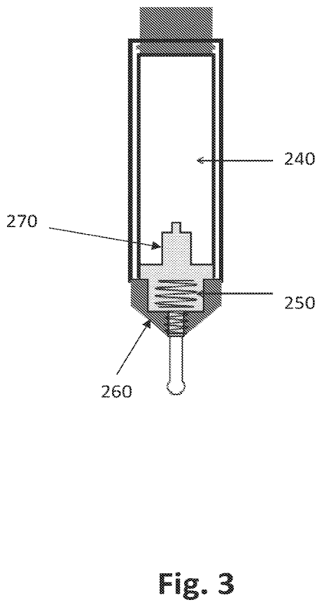

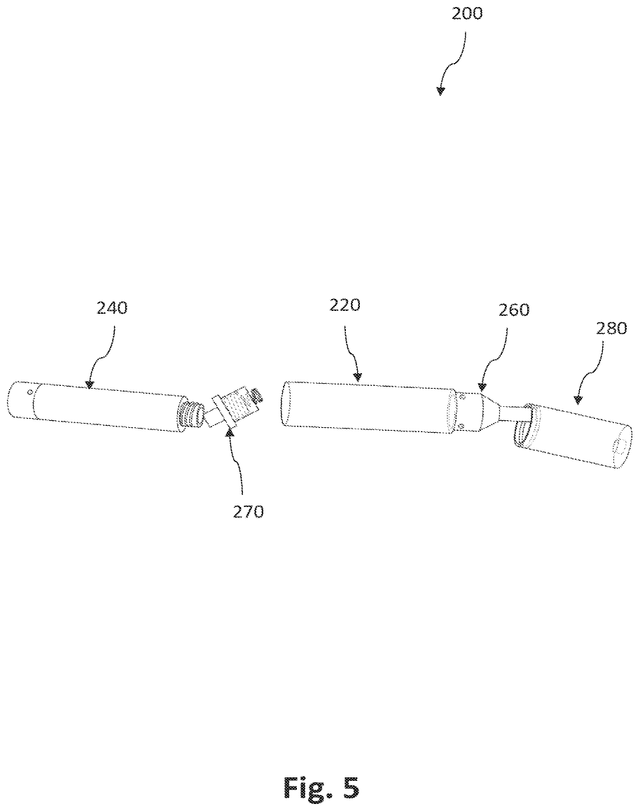

FIG. 2 shows an example of packing of the serum. The packaging 200 has a long tubular nozzle 230 through which the liquid serum can be dispensed dropwise. The flow of liquid serum and the formation of droplets of equal sizes can be affected by the long tubular nozzle 230. The packaging has a tubular body 220, which may contain liquid serum. The proximal end of the tubular body 220 has the long tubular nozzle 230, and the distal end may have an actuator 210 that can dispense the liquid serum as droplets through the long tubular nozzle 230. The actuator 210 may have a button that can be depressed to dispense the serum. FIG. 3 shows the inner components of the serum dispenser 200. The serum dispenser 200 may include a chamber 240 within the tubular body 220, wherein the chamber 240 may contain the liquid serum. The actuation mechanism may include two sets of spring coils. The nozzle 230, part 260, and the tubular body 220 can be integral and separated to expose the part 270. The part 270, which includes the actuation mechanism can be screwed to the chamber 240. The part 270 can be removed to expose an opening of the chamber 240. Liquid serum can be filled into the chamber through the opening. Parts 260 and 270 are further illustrated in the exploded view shown in FIG. 5. A cap 280 may be secured to the part 260.

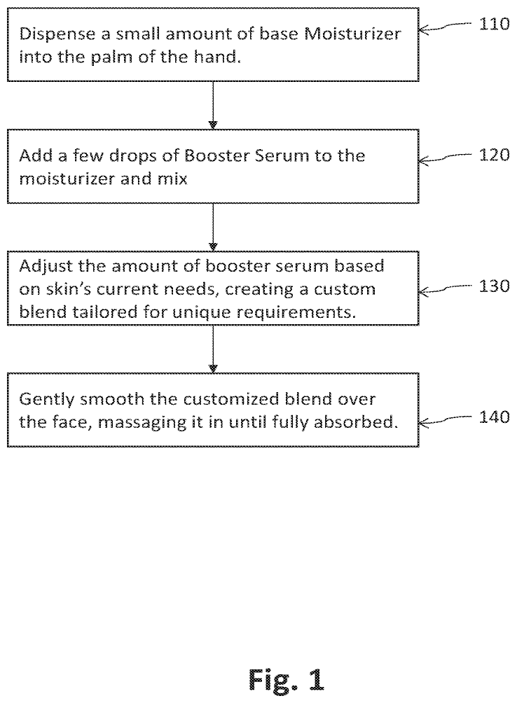

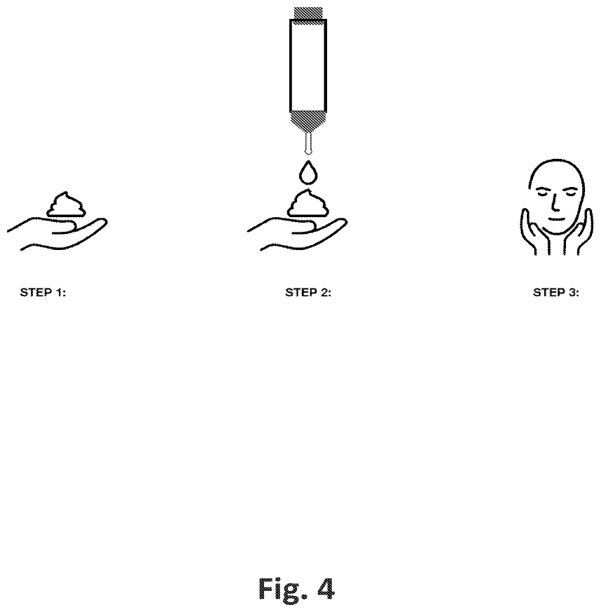

Referring to FIGS. 1 and 4, which illustrate the process of using the disclosed skin care system. First, a user can dispense a measured portion of the base moisturizer (e.g., a “penny-sized” amount) into the palm of the hand or another mixing surface, at step 110. The user can then select one or more functional Booster Serums appropriate for their current skin condition or desired outcome. The user dispenses a predetermined number of drops of the chosen functional Booster Serum(s) directly into the base moisturizer at step 120. If needed, additional drops of serum can be added at step 130. The serum can be gently blended with moisturizer, for example, using fingers, creating a homogenous mixture. The user may then apply the customized formulation to the skin (e.g., the face or other areas) at step 140. The base moisturizer may serve as a carrier and delivery medium for the functional booster serums. The functional booster serums provide concentrated active ingredients targeting specific skin functions. The customizable ratio of base moisturizer to functional Booster Serum(s) empowers the user to control both the intensity and function of the skincare treatment in real time.

Thus, the disclosed skin care system allows controlling both the type and quantity of functional Booster Serum(s) added to the base moisturizer. The user can create a unique formulation tailored to their specific skincare needs at each application. The disclosed system allows for daily, seasonal, or situational adjustments, accommodating fluctuations in skin condition that conventional skincare products cannot address. The process promotes user engagement, customization, and efficacy, representing an advancement in personalized skincare.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

1. A method of preparing a customized skin care formulation at a time of application, comprising:
dispensing a measured portion of a base moisturizer from a base moisturizer dispenser onto a user-accessible mixing surface; dispensing, from a booster serum dispenser separate from the base moisturizer dispenser, a user-selected number of discrete liquid droplets of a booster serum directly onto the dispensed base moisturizer; manually blending the base moisturizer and the dispensed droplets of the booster serum immediately prior to application to form a homogeneous mixture; and applying the homogeneous mixture to skin, wherein the base moisturizer functions as a carrier for the booster serum, and wherein a concentration of one or more active ingredients delivered to the skin is determined at the time of application by the number of droplets dispensed by the user.

2. A skin care system comprising:
a base moisturizer dispenser configured to dispense a predetermined quantity of a base moisturizer onto a user-accessible mixing surface; and
a booster serum dispenser separate from the base moisturizer dispenser and configured to dispense discrete liquid droplets of a booster serum directly onto the dispensed base moisturizer, wherein the system is configured such that a user manually blends the base moisturizer and the booster serum immediately prior to application to skin, and wherein a concentration of one or more active ingredients delivered to the skin is adjustable at each application by varying a number of droplets dispensed by the booster serum dispenser.

3. The method of claim 1, wherein the booster serum dispenser is configured to dispense droplets of substantially uniform volume.

4. The method of claim 1, wherein the booster serum dispenser includes a tubular nozzle configured to dispense the booster serum dropwise.

5. The method of claim 1, wherein no formulation recipe is generated, stored, or executed by a machine prior to dispensing the base moisturizer or the booster serum.

6. The method of claim 1, wherein the homogeneous mixture is applied to the skin during the same application session in which the base moisturizer and the booster serum are dispensed.

7. The method of claim 1, wherein the base moisturizer is free of skin-treatment active ingredients and serves as a universal carrier for different booster serums.

8. The method of claim 1, wherein the booster serum is selected by the user based on a current skin condition at the time of application.

9. The method of claim 1, wherein manually blending comprises blending the base moisturizer and the booster serum using a user's fingers on the mixing surface.
